(19) United States
(12) Patent Application Publication
(10) Pub. No.: US 2026/0265756 A1
(43) Pub. Date: Sep. 10, 2026

(54) VPS4B INHIBITOR FOR USE IN METHODS FOR THE TREATMENT OF HRD CANCER

(71) Applicants: INSTITUT CURIE, PARIS (FR); INSTITUT NATIONAL DE LA SANTE ET DE LA RECHERCHE MEDICALE, PARIS (FR)

(72) Inventors: Raphaël CECCALDI, PARIS (FR); Marie VALLETTE, ORLEANS (FR); Marton KOVACS, SCEAUX (FR)

(21) Appl. No.: 19/165,226

(22) PCT Filed: Mar. 21, 2024
(86) PCT No.: PCT/EP2024/057558

§ 371 Date: Sep. 15, 2025

(30) Foreign Application Priority Data
Mar. 21, 2023 (EP) 23305380.0

(51) Int. Cl. C12N 15/113; A61K 31/7048; A61K 33/243; A61K 45/06; A61P 35/00; C12N 15/115

(52) U.S. Cl. CPC C12N 15/1137; A61K 31/7048; A61K 33/243; A61K 45/06; A61P 35/00; C12N 15/115; C12N 2310/11; C12N 2310/14; C12N 2310/16; C12N 2310/531; C12Y 306/04006

(57) ABSTRACT

The invention relates to methods and pharmaceutical compositions for the treatment of HRD cancer, particularly BRCA-associated cancer and chemo-resistance BRCA-associated cancer. The inventors investigated the role of VPS4B in HRD cancer, particularly in BRCA-associated cancer. The inventors demonstrated that HR-deficient cells have NE vulnerability, increased NE rupture and are sensitive to inhibitor of NE repair factors. The inventors also demonstrated that VPS4B is synthetic lethal in BRCA1/2-mutated cells and indicate that BRCA2−/− cells were extremely sensitive to VPS4B inhibition. Altogether, the present invention highlights the role of nuclear envelope (NE) repair factors, particularly VPS4B inhibitors in HRD cancer and the use of VPS4B inhibitors in the treatment of HRD cancer, particularly BRCA-associated cancer and BRCA-associated cancer with acquired drug resistance. Thus, the present invention relates to VPS4B inhibitor for use in the treatment of HRD cancer, particularly BRCA-associated cancer and chemo-resistance BRCA-associated cancer.

### FIELD OF THE INVENTION

The invention relates to methods and pharmaceutical compositions for the treatment of cancer, particularly HRD cancer, BRCA-associated cancer and chemo-resistance BRCA-associated cancer.

### BACKGROUND OF THE INVENTION

In the last decades, many efforts have been undertaken to gain mechanistic insights into the synthetic lethality between PARP1 and BRCA1/2 genes (Maya-Mendoza et al., 2018; Hanzlikova et al., 2018). Deficiencies in homologous recombination (HR)-mediated DNA repair occur mainly through genetic inactivation of BRCA1 and BRCA2 (BRCA1/2) genes and play a role in the initiation and progression of many tumor types. Defective HR (HRD) causes genomic instability and hyper-dependence on alternative DNA repair mechanisms for survival, setting the stage for synthetic lethality-based targeted therapy, as exemplified by the extreme sensitivity of HRD tumors to poly (ADP-ribose) polymerase inhibitors (PARPi) (Bryant et al., 2005; Farmer et al., 2005). This lethal interaction is successfully exploited in the clinic since the approval of PARP inhibitors (PARPi) for the treatment of BRCA1/2-mutated tumors (Patel et al., 2021), also referred as homologous recombination (HR)-deficient tumors (HRD). Various trials have been devised to evaluate PARPi effectiveness for patients with breast, ovarian, and prostate tumors harboring BRCA1/2 mutations (Pujade-Lauraine et al., 2017). However, PARPi and other chemotherapeutics have shown limited effectiveness in achieving HRD cancer remission, notably because drug resistance emerges and resistance to chemotherapy is emerging as the major obstacle to clinic effectiveness (Gogola et al., 2019). Hence, no therapeutic options are left for these patients, stressing the need for alternative therapeutic options (Konstantinopoulos et al., 2015).

The inventors investigated new mechanism of synthetic lethal interaction of nuclear envelope (NE) rupture factors with BRCA1/2 proteins to find alternative curative options for HRD cancer including BRCA1/2-mutated tumors, and the ones that have acquired PARPi resistance. The inventors demonstrated that HR-deficient cells have NE vulnerability, increased NE rupture and are sensitive to inhibitor of NE repair factors. The inventors discovered a new synthetic lethal interaction between nuclear envelope (NE) rupture factors and BRCA1/2 proteins and show particularly that the VPS4B is synthetic lethal in BRCA1/2-mutated cells.

The inventors identified DNA-damage as a source of nuclear envelope (NE) rupture. To understand the implications for cancer cells that have endogenous DNA damage, the inventors knocked out BRCA2 in RPE1, TP53−/− cells. Using these isogenic line models, the inventors found that, compared to the modest levels of NE rupture observed in the WT cells, BRCA2−/− cells showed a 3-fold increase in NE rupture. To further expand on these findings, the inventors wanted to explore the consequences of NE rupture in HR-deficient tumors. First, the inventors confirmed in several in HR-proficient and -deficient tumor cell lines that HR-deficiency leads to an increased level of NE rupture. The inventors found that, in RPE model system, knockdown of LaminA/C led to a higher increase in NE rupture in BRCA2−/− cells compared to the WT cells. Furthermore, the level of NE rupture correlated well with cell survival. The inventors found that, while WT cells suffered slightly from Lamin A/C knockdown, BRCA2−/− cells were extremely sensitive, with almost no surviving colonies upon siRNA treatment. The inventors further validated this by using siLaminA/C in panel of HR-proficient and -deficient cancer cells. Similarly to RPE model system, HR-deficient cancer cells showed an increased sensitivity to LaminA/C knockdown.

Here, to find alternative curative options for BRCA1/2-mutated tumors, and the ones that have acquired PARPi resistance, the inventors investigated the role of VPS4B in cancer, particularly in BRCA-associated cancer and PARPi resistance. The inventors show that VPS4B is synthetic lethal with BRCA2. Interestingly, the inventors found that BRCA2−/− cells were extremely sensitive to knockdown of VPS4B, a component of the ESCRTIII complex, responsible for NE reassembly and repair (Christ et al., 2017). Similarly, VPS4B−/− cells were extremely sensitive to BRCA2 knockdown, confirming the synthetic lethal relationship between the two proteins. This indicates that HR-deficient cells have an intrinsic NE vulnerability, resulting in NE rupture and defective NE reassembly. In line with this evidence, when observing mitotic exit using live microscopy, the inventors found that while deficiency in either BRCA2 or VPS4B led to an increase in the frequency of mitotic catastrophe, loss of both factors led to a synergistic effect, resulting in extensive mitotic abnormalities and cell death. The inventors confirmed that mitotic catastrophe upon VPS4B knockdown is not due to DNA damage accumulation, as they saw no increase in the number of γH2AX foci in these conditions.

The results indicate that HR-deficient cells rely on proper lamina organization for their survival, reinforcing the notion of increased NE vulnerability in these cells. Based on these findings, the inventors propose that targeting NE vulnerability in these cells by either weakening the lamina or disrupting NE repair or reformation mechanisms, is a novel avenues for the treatment of HR-deficient tumors. Additionally, the mechanism of action being completely different than traditional DNA damage-based chemotherapeutics, targeting NE vulnerability could be used as a complimentary or alternative therapy to current treatments. Furthermore, the inventors indicate that HR-deficient tumors with acquired chemoresistance still display NE vulnerability, as they rely heavily on ATR activity for their survival (Yazinski et al., 2017).

There is no disclosure in the art of the role of VPS4B inhibitor in cancer, particularly Homologous recombination deficiency (HRD) cancer and BRCA-associated cancer, and the targeting of VPS4B in the treatment of cancer, particularly HRD cancer and BRCA-associated cancer.

### SUMMARY OF THE INVENTION

The invention relates to methods and pharmaceutical compositions for the treatment of cancer, particularly HRD cancer and BRCA-associated cancer. In particular, the invention is defined by the claims.

### DETAILED DESCRIPTION OF THE INVENTION

The inventors investigated the role of new mechanism of synthetic lethal interaction between nuclear envelope (NE) rupture factors with BRCA1/2 proteins to find alternative curative options for HRD cancer including BRCA1/2-mutated tumors, and the ones that have acquired PARPi resistance. The inventors demonstrated that HR-deficient cells have NE vulnerability, increased NE rupture and are sensitive to inhibitor of NE repair factors. The inventors also demonstrated that VPS4B is synthetic lethal in BRCA1/2-mutated cells and indicate that BRCA2−/− cells were extremely sensitive to VPS4B inhibition. Altogether, the present invention highlights the role of nuclear envelope (NE) repair factors, particularly VPS4B inhibitors in cancer and the use of VPS4B inhibitors in the treatment of cancer, particularly HRD cancer including BRCA-associated cancer and BRCA-associated cancer with acquired drug resistance in mono-or combination therapy with PARPi.

Accordingly, the invention relates to the targeting of VPS4B in the treatment of cancer, particularly HRD cancer, BRCA-associated cancer and chemo-resistance BRCA-associated cancer.

### Therapeutic Method

Accordingly, in a first aspect, the invention relates to a VPS4B inhibitor for use in the treatment of cancer.

In some embodiment, the invention relates to VPS4B inhibitor for use in the treatment of HRD cancer.

In some embodiment, the invention relates to VPS4B inhibitor for use in the treatment of BRCA-associated cancer.

In some embodiment, the invention relates to VPS4B inhibitor for use in the treatment of BRCA-associated cancer, particularly chemo-resistance BRCA-associated cancer such as PARPi resistant BRCA-associated cancer or cisplatin resistant BRCA-associated cancer.

As used herein, the terms “subject”, “individual” or “patient” are interchangeable and refer to a mammal. Typically, a subject according to the invention refers to any subject, preferably human. In a particular embodiment, the term “subject” refers to a subject afflicted or at risk to be afflicted with cancer. In a particular embodiment, the term “subject” refers to a subject afflicted or at risk to be afflicted with HRD cancer. In a particular embodiment, the term “subject” refers to a subject afflicted or at risk to be afflicted with BRCA-associated cancer. In some embodiment, the term “subject” refers to a subject afflicted or at risk to be afflicted with chemo-resistance BRCA-associated cancer. In some embodiment, the term “subject” refers to a subject afflicted or at risk to be afflicted with BRCA-associated cancer such as Homologous recombination deficiency (HRD) and/or BRCA-deficiency (basal-like, luminal, and HER2-overexpressing breast carcinomas and other cancers) and breast, ovarian, prostate, pancreatic or any other type of tumors harboring BRCA1/2 mutations.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarninoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Karposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Karposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

The term “homologous recombination deficiency cancer” or “HRD cancer” has its general meaning in the art and refers to cancer displaying defective homologous recombination (HRD)-mediated DNA repair which causes genomic instability and hyper-dependence on alternative DNA repair mechanisms for survival. Thus, the term “HRD cancer” refers to a cancer displaying defective homologous recombination due to alteration of genes including PALB2, CHEK2, ATM, BARD1, RAD51C, RAD51D, FANCC, BRIP1, FANCM, XRCC2, or any other genes involved in HR DNA repair pathway. The HR status of a cancer can be determined by any well-known methods in the art, for example by probing the genome for HRD-related genomic signatures or by using a commercial HRD diagnostic test such as Myriad myChoice®. The term “HRD cancer” include but is not limited to BRCA-associated cancer. Conversely, a BRCA-associated cancer is not necessarily a HRD cancer.

The term “BRCA-associated cancer” has its general meaning in the art and refers to cancer associated with BRCA mutation or BRCA expression deficiency. The term “BRCA-associated cancer” refers to cancer selected from cancer associated with BRCA1 and/or BRCA2 mutation, in particular inactivation of BRCA1 and/or BRCA2 genes, cancer associated with BRCA1 and/or BRCA2 expression deficiency, and homologous recombination deficiency (HRD) cancer with BRCA-deficiency (such as basal-like, luminal, and HER2-overexpressing carcinomas, breast, ovarian, and prostate tumors harboring BRCA1/2 mutations and other cancers). The term “BRCA-associated cancer” does not refer to a cancer with a functional BRCA1/2. In some embodiment, the term “BRCA-associated cancer” refers to breast cancer, ovary cancer, cervix cancer, pancreas cancer, lung cancer, head and neck cancer and melanoma with BRCA1 and/or BRCA2 mutation or BRCA1 and/or BRCA2 expression deficiency.

As used herein “BRCA1/2” denotes BRCA1 and/or BRCA2, more particularly BRCA1 and BRCA2.

The term “BRCA-associated cancer” also refers to chemo-resistance BRCA-associated cancer such as PARP inhibitor (PARPi) resistant BRCA-associated cancer, PARPi-resistant HRD tumors including tumors with somatic reversion of BRCA1/2 mutation and subsequent HR restoration, cisplatin resistant BRCA-associated cancer and cisplatin-resistant BRCA1 and BRCA2-mutated tumors including tumors with somatic reversion of BRCA1/2 mutation and subsequent HR restoration.

The term “PARP inhibitor” or “PARPi” has its general meaning in the art and refers to PARP inhibitor such as olaparib, rucaparib, niraparib and talazoparib. The term “PARP inhibitor” also refers to PARP inhibitor such iniparib, veliparib, Pamiparib (BGB-290), CEP 9722, E7016 and 3-Aminobenzamide.

The term “VPS4B” has its general meaning in the art and refers to a Vacuolar Protein Sorting 4 Homolog B also known as Vacuolar Protein Sorting-Associated Protein 4B (the sequence of human VPS4B Protein is available in UniProtKB/Swiss-Prot under Accession number O75351, release number 2023_01/2023_01: version of 22 Feb. 2023; mRNA sequence available in Genbank under accession number AF195514.1). VPS4B is also known as SKD1 or VPS42.

As used herein, the term “VPS4B inhibitor” refers to any compound selected from the group consisting of but not limited to compounds targeting Vacuolar Protein Sorting 4 Homolog B. The term “VPS4B inhibitor” refers to compounds that bind to VPS4B and function as potent antagonists of VPS4B. The term “VPS4B inhibitor” has its general meaning in the art and refers to a compound that selectively inactivates VPS4B. Typically, a VPS4B inhibitor is a small organic molecule, a polypeptide, an aptamer, an oligonucleotide (antisense oligonucleotides, siRNA, shRNA, DNA and RNA aptamers), or an antibody. VPS4B inhibitors are well-known in the art as such as described in Frey et al., 2022; Szymańska et al., 2020.

The term “VPS4B inhibitor” refers to any compound selected from but not limited to siRNA such siVPS4B #1 and siVPS4B #2 of the example and siVPS4B #1 (s18272, targeting exon 7 of VPS4B mRNA, at position 961 of Genbank mRNA AF195514.1), siVPS4B #2 (s18273, targeting exons 8, 9 of VPS4B mRNA, at position 1076 of Genbank mRNA AF195514.1), siVPS4B #3 (s231857, targeting exon 6 of VPS4B mRNA, at position 803 of Genbank mRNA AF195514.1) and siVPS4B #4 (s231858, targeting exon 3 of mouse VPS4B mRNA, at position 429 of Genbank mRNA AK151023.1) described in Szymańska et al., 2020.

“VPS4B inhibitor” can also refer to antibodies, microRNA such as miR-488-3p, short hairpin VPS4B (sh-VPS4B), or subunits from VPS4B dominant negative mutants such as VPS4B-K180Q or mutants with E to Q mutation at amino acid residue 235.

Small organic molecule inhibitors of VPS4B include for instance MSC1094308 (CAS No.: 2219320-08-6), and ATP-competitive inhibitor DBeQ (CAS No.: 177355-84-9).

Tests and assays for determining whether a compound is a VPS4B inhibitor are well known by the skilled person in the art such as described in Frey et al., 2022; Szymańska et al., 2020. Determining whether a compound is a VPS4B inhibitor may also be performed using by using recombinant VPS4B proteins, competitive binding assays and measuring the binding affinities, measuring synthetic lethal interaction between VPS4B and BRCA1/2 proteins or assays such as described in the examples.

In another embodiment, the compound of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the target of the invention as above described, the skilled man in the art can easily select those blocking or inactivating the target.

In another embodiment, the compound of the invention is an antibody (the term including “antibody portion”) directed against the target.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria, which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGl, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′)2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGl, IgG2, IgG3 and IgG4. In a preferred embodiment, the compound of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals, which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In another aspect, the invention provides an antibody that competes for binding to the target with the antibody of the invention.

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, NJ) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard antigen binding assays. The ability of a test antibody to inhibit the binding of antibodies of the present invention to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the invention provides antibodies that bind to the same antigen as, and compete with, the antibodies disclosed herein. As used herein, an antibody “competes” for binding when the competing antibody inhibits the target binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of the target. In some embodiments, the epitopes to which the present antibodies or antigen binding fragments bind are linear epitopes. In other embodiments, the epitopes to which the present antibodies or antigen binding fragments bind are non-linear, conformational epitopes.

In one embodiment, the VPS4B inhibitor of the invention is a VPS4B expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene. An “inhibitor of expression” refers to any compound that has a biological effect to inhibit the expression of a target gene and/or the expression of target protein. In one embodiment of the invention, said inhibitor of expression is a short hairpin RNA (shRNA), a small inhibitory RNA (siRNA), or an antisense oligonucleotide. Preferably, the inhibitor of expression is a siRNA or a shRNA.

The target expression inhibitors for use in the present invention may be based on antisense oligonucleotide constructs. Anti-sense oligonucleotides, including anti-sense RNA molecules and anti-sense DNA molecules, would act to directly block the translation of the target mRNA by binding thereto and thus preventing protein translation or increasing mRNA degradation, thus decreasing the level of the target proteins, and thus activity, in a cell. For example, antisense oligonucleotides of at least about 15 bases and complementary to unique regions of the mRNA transcript sequence encoding the target can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as a target expression inhibitors for use in the present invention. The target gene expression can be reduced by contacting the subject or cell with a small double stranded RNA (dsRNA), or a vector or construct causing the production of a small double stranded RNA, such that the target expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Short hairpin RNA (shRNA) or Small inhibitory RNAs (siRNAs) can function as inhibitors of gene expression for use in the invention. Gene expression can be reduced with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known.

Ribozymes can also function as target expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the target mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides (ODNs) and ribozymes useful as target inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the target. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol.7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a further aspect, the invention relates to a method of treating HRD cancer in a subject in need thereof, comprising administering to the subject a therapeutically effective amount of a VPS4B inhibitor.

In some embodiments, the invention relates to a method of treating BRCA-associated cancer in a subject in need thereof, comprising administering to the subject a therapeutically effective amount of a VPS4B inhibitor.

In some embodiments, the invention relates to a method of treating BRCA-associated cancer, particularly chemo-resistance BRCA-associated cancer such as PARPi resistant BRCA-associated cancer or cisplatin resistant BRCA-associated cancer in a subject in need thereof, comprising administering to the subject a therapeutically effective amount of a VPS4B inhibitor.

In some embodiments, the VPS4B inhibitor and/or pharmaceutical composition according to the invention is used in combination with PARP inhibitor (PARPi).

In some embodiment, the PARP inhibitor is selected from the group consisting of olaparib, rucaparib, niraparib, talazoparib, iniparib, veliparib, Pamiparib (BGB-290), CEP 9722,E7016, E7449 and 3-Aminobenzamide.

In some embodiments, the VPS4B inhibitor and/or pharmaceutical composition according to the invention is used in combination with cisplatin.

In some embodiments, the VPS4B inhibitor and/or pharmaceutical composition according to the invention is used in combination with Pole inhibitors such as novobiocin.

In some embodiment, the VPS4B inhibitor and/or pharmaceutical composition according to the invention is used in combination with a NMNAT1 inhibitor.

As used herein, the term “NMNAT1 inhibitor” refers to any compound selected from the group consisting of but not limited to compounds targeting Nicotinamide Nucleotide Adenylyltransferase 1. The term “NMNAT1 inhibitor” refers to compounds that bind to NMNAT1 and function as potent antagonists of NMNAT1. The term “NMNAT1 inhibitor” has its general meaning in the art and refers to a compound that selectively inactivates NMNAT1. Typically, a NMNAT1 inhibitor is a small organic molecule, a polypeptide, an aptamer, an oligonucleotide (antisense oligonucleotides, siRNA, shRNA, DNA and RNA aptamers), or an antibody. NMNAT1 inhibitors are well-known in the art as such as described in WO2013/130672; Kusumanchi et al., 2013.

The term “NMNAT1 inhibitor” refers to any compound selected from but not limited to siRNA such s34981 (hNMNAT1 siRNA) (Kusumanchi et al., 2013; WO2013/130672), siRNA ID130437, siRNA ID130438, siRNA ID130439, siRNA ID212976, siRNA ID212977 (Thermo Fisher Scientific).

In some embodiment, the VPS4B inhibitor and/or pharmaceutical composition according to the invention is used in combination with a SIRT6 inhibitor.

In some embodiment, said SIRT6 inhibitor is selected from the group consisting of OSS128167, Trichostatin A and SIRT6 inhibitory Quercetin derivatives luteolin, catechin gallate and gallocatechin gallate.

In some embodiments, the VPS4B inhibitor and/or pharmaceutical composition according to the invention is used in combination with a compound selected from the group consisting of PARP inhibitor (PARPi), NMNAT1 inhibitor, SIRT6 inhibitor and/or Pole inhibitors.

In some embodiments, the VPS4B inhibitor and/or pharmaceutical composition according to the invention is used in combination with cancer therapies. In particular, compound and/or pharmaceutical composition of the invention may be administered in combination with targeted therapy, immunotherapy such as immune checkpoint therapy and immune checkpoint inhibitor, co-stimulatory antibodies, chemotherapy and/or radiotherapy.

As used herein, the term “immunotherapy” refers to a cancer therapeutic treatment using the immune system to reject cancer. The therapeutic treatment stimulates the patient's immune system to attack the malignant tumor cells.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B-and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Immune checkpoint therapy also include co-stimulatory antibodies delivering positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, atezolizumab, durvalumab and avelumab. Example of anti-CTLA-4 antibodies include, but are not limited to, ipilimumab (see, e.g., U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., International patent applications WO1997020574 and WO2007123737) and antibodies described in U.S. Pat. No. 8,491,895. Example of anti-VISTA antibodies are described in US patent application US20130177557. Example of inhibitors of the LAG3 receptor are described in U.S. Pat. No. 5,773,578. Example of KIR inhibitor is IPH4102 targeting KIR3DL2.

In some embodiments, the compound and/or pharmaceutical composition of the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the compound and/or pharmaceutical composition of the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the compound and/or pharmaceutical composition of the invention is administered to the subject in combination with radiotherapy. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun 1;104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov 7;8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015;31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul 11).

### Pharmaceutical Composition

The compounds of the invention may be used or prepared in a pharmaceutical composition.

In one embodiment, the invention relates to a pharmaceutical composition comprising the compound of the invention and a pharmaceutical acceptable carrier for use in the treatment of cancer in a subject of need thereof.

In some embodiments, the invention relates to a pharmaceutical composition comprising the compound of the invention and a pharmaceutical acceptable carrier for use in the treatment of HRD cancer in a subject in need thereof.

In some embodiments, the invention relates to a pharmaceutical composition comprising the compound of the invention and a pharmaceutical acceptable carrier for use in the treatment of BRCA-associated cancer in a subject in need thereof.

In some embodiment, the invention relates to a pharmaceutical composition comprising the compound of the invention and a pharmaceutical acceptable carrier for use in the treatment of BRCA-associated cancer, particularly chemo-resistance BRCA-associated cancer such as PARPi resistant BRCA-associated cancer or cisplatin resistant BRCA-associated cancer.

Typically, the compound of the invention may be combined with pharmaceutically acceptable excipients, and optionally sustained-release matrices, such as biodegradable polymers, to form therapeutic compositions.

Typically, the compounds according to the invention as described above are administered to the subject in a therapeutically effective amount.

By a “therapeutically effective amount” of the compound of the present invention as above described is meant a sufficient amount of the compound at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5 1.0, 2.5, 5.0, 10.0, 15.0, 25.0, 50.0, 100, 250 and 500 mg of the compound of the present invention for the symptomatic adjustment of the dosage to the patient to be treated. A medicament typically contains from about 0.01 mg to about 500 mg of the compound of the present invention, preferably from 1 mg to about 100 mg of the compound of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the compound according to the invention may be used in a concentration between 0.01 μM and 20 μM, particularly, the compound of the invention may be used in a concentration of 0.01, 0.05, 0.1, 0.5, 1.0, 2.5, 5.0, 10.0, 15.0, 20.0 μM.

According to the invention, the compound of the present invention is administered to the subject in the form of a pharmaceutical composition. Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agents of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the compound of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

Pharmaceutical compositions of the invention may include any further compound which is used in the treatment of cancer such as described above.

In some embodiments, the pharmaceutical compositions of the invention may include any further compound which is used in the treatment of BRCA-associated cancer.

In one embodiment, said additional active compounds may be contained in the same composition or administrated separately.

In another embodiment, the pharmaceutical composition of the invention relates to combined preparation for simultaneous, separate or sequential use in the treatment of cancer in a subject in need thereof.

In some embodiments, the pharmaceutical composition of the invention relates to combined preparation for simultaneous, separate or sequential use in the treatment of BRCA-associated cancer in a subject in need thereof.

The invention also provides kits comprising the compound of the invention. Kits containing the compound of the invention find use in therapeutic methods.

The invention will be further illustrated by the following figures and examples. However, these examples and figures should not be interpreted in any way as limiting the scope of the present invention.

### FIGURES

FIG. 1: HR-deficient cells have increased NE rupture and are sensitive to Lamin A/C knockdown.

A, Quantification of NE rupture upon Lamin A/C knockdown in WT and BRCA2−/− cells. B, Quantification of clonogenic survival upon Lamin A/C knockdown Survival was normalized to siCtrl in each experiment, for each cell line. C, Quantification of NE rupture in a panel of RPE1 and cancer cells. D, Quantification of clonogenic survival upon Lamin A/C knockdown Survival was normalized to siCtrl in each experiment, for each cell line. Data shows mean +/− S.E.M.

FIG. 2: VPS4B is synthetic lethal with BRCA2

A-B, Quantification of clonogenic potential of WT, BRCA2−/− and VPS4B−/− cells cells upon indicated siRNA treatment. Survival was normalized to siCtrl in each experiment, for each cell line. Representative images are shown on the right. C, Quantification of mitotic catastrophe in WT and BRCA2−/− cells upon indicated siRNA treatment. On the right, still images from representative time lapse imaging of mitotic catastrophe upon VPS4B knockdown in BRCA2−/− cells. Time after mitotic entry is shown on images (in minutes) A total of at least 150 mitoses were analyzed among 3 experiments for each condition.

### EXAMPLE

### HR-Deficient Cells Have NE Vulnerability

### Results

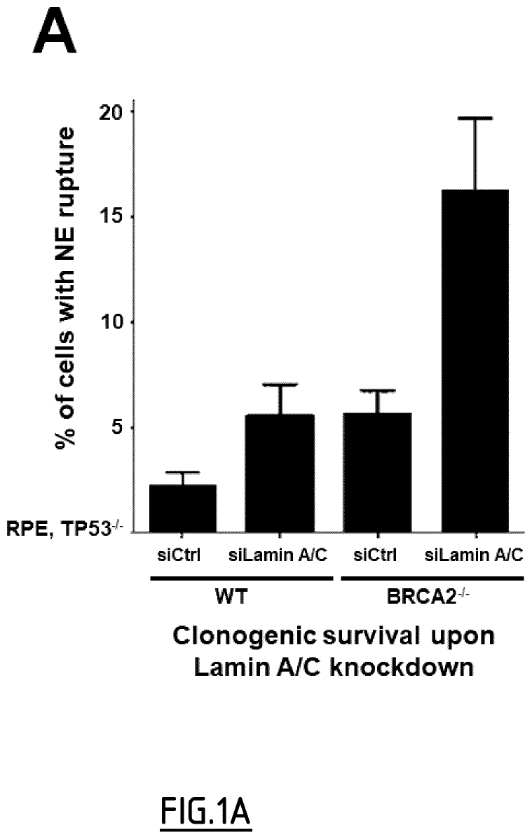

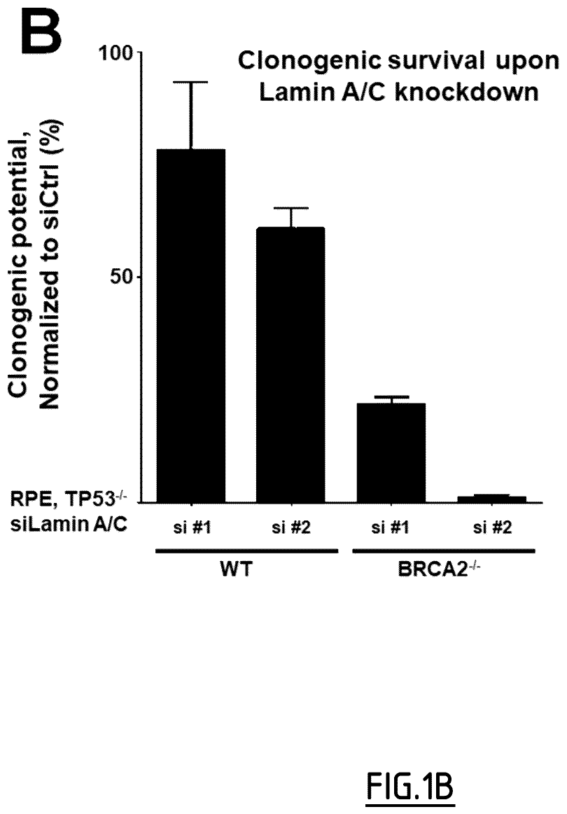

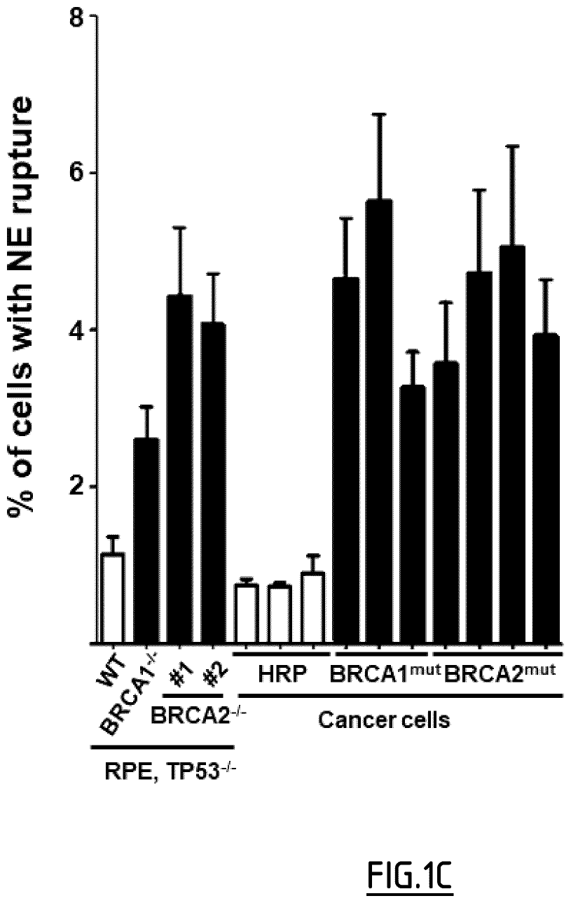

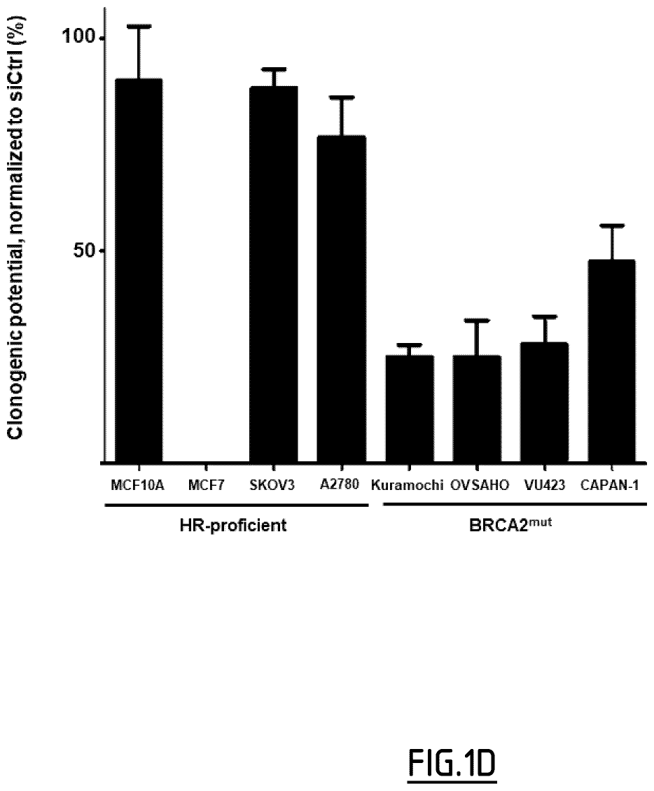

### HR-Deficient Cells Have Increased NE Rupture and Are Sensitive to Lamin A/C Knockdown (FIG. 1)

The inventors identified DNA-damage as a source of nuclear envelope (NE) rupture. Upon DNA damage the DDR kinase ATR gets activated, ultimately leading to NE rupture. To understand the implications for cancer cells that have endogenous DNA damage, the inventors knocked out BRCA2 in RPE1, TP53−/− cells. Using these isogenic line models, the inventors found that, compared to the modest levels of NE rupture observed in the WT cells, BRCA2−/− cells showed a 3-fold increase in NE rupture. As NE rupture events are generally transient, to have a more accurate measurement of the frequency of these events, the inventors decided to do live microscopy, following icGAS-GFP to monitor NE rupture events. The inventors found that a striking 27% of BRCA2−/− cells underwent at least one event of NE rupture during a full cell cycle. This high frequency underscores the need to better understand this phenomenon.

To further expand on these findings, the inventors wanted to explore the consequences of NE rupture in HR-deficient tumors. First, the inventors confirmed in several in HR-proficient and -deficient tumor cell lines that HR-deficiency leads to an increased level of NE rupture. Given this baseline difference in NE integrity, the inventors wondered what would happen if they pushed the system further by knocking down Lamin A/C. The inventors found that, in RPE model system, knockdown of LaminA/C led to a higher increase in NE rupture in BRCA2−/− cells compared to the WT cells. Furthermore, the level of NE rupture correlated well with cell survival. The inventors found that, while WT cells suffered slightly from Lamin A/C knockdown, BRCA2−/− cells were extremely sensitive, with almost no surviving colonies upon siRNA treatment. The inventors further validated this by using siLaminA/C in our panel of HR-proficient and -deficient cancer cells. Similarly to RPE model system, HR-deficient cancer cells showed an increased sensitivity to LaminA/C knockdown. A notable exception to this was the HR-proficient MCF7 cell line, which was extremely sensitive. This could be due to other, unknown structural vulnerabilities of these cells, for example changes in cytoskeleton organization.

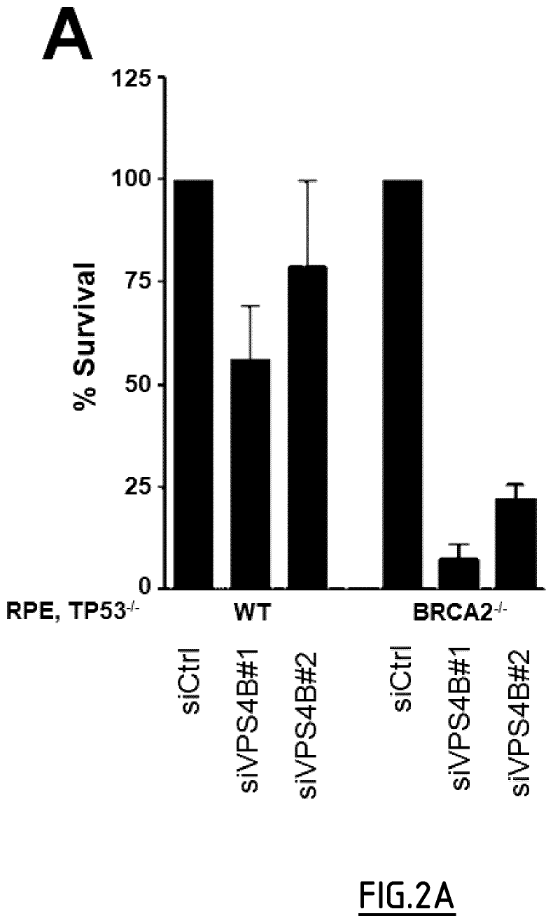

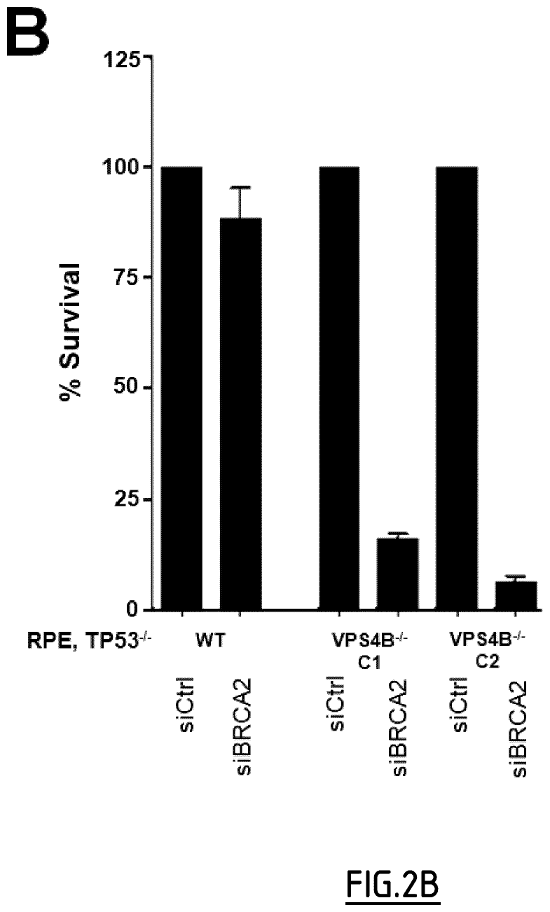

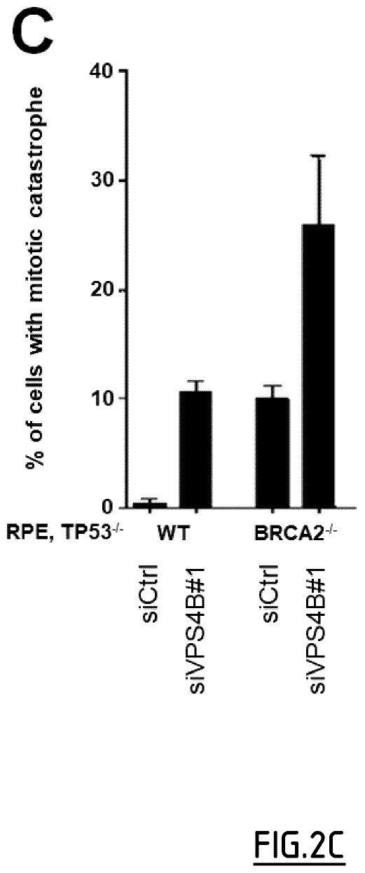

### VPS4B is Synthetic Lethal with BRCA2 (FIG. 2)

Interestingly, the inventors found that BRCA2−/− cells were extremely sensitive to knockdown of VPS4B, a component of the ESCRTIII complex, responsible for NE reassembly and repair (Christ et al., 2017). Similarly, VPS4B−/− cells were extremely sensitive to BRCA2 knockdown, confirming the synthetic lethal relationship between the two proteins. This indicates that HR-deficient cells have an intrinsic NE vulnerability, resulting in NE rupture and defective NE reassembly. In line with this evidence, when observing mitotic exit using live microscopy, the inventors found that while deficiency in either BRCA2 or VPS4B led to an increase in the frequency of mitotic catastrophe, loss of both factors led to a synergistic effect, resulting in extensive mitotic abnormalities and cell death. The inventors confirmed that mitotic catastrophe upon VPS4B knockdown is not due to DNA damage accumulation, as the inventors saw no increase in the number of γH2AX foci in these conditions.

The results indicate that HR-deficient cells rely on proper lamina organization for their survival, reinforcing the notion of increased NE vulnerability in these cells.

Based on this findings, the inventors propose that targeting NE vulnerability in these cells by either weakening the lamina or disrupting NE repair or reformation mechanisms, is a novel avenues for the treatment of HR-deficient tumors. Additionally, the mechanism of action being completely different than traditional DNA damage-based chemotherapeutics, targeting NE vulnerability could be used as a complimentary or alternative therapy to current treatments. Furthermore, the inventors posit that HR-deficient tumors with acquired chemoresistance still display NE vulnerability, as they rely heavily on ATR activity for their survival (Yazinski et al., 2017).

### REFERENCES

Throughout this application, various references describe the state of the art to which this invention pertains. The disclosures of these references are hereby incorporated by reference into the present disclosure.
Bryant, H. E.; Schultz, N.; Thomas, H. D.; Parker, K. M.; Flower, D.; Lopez, E.; Kyle, S.; Meuth, M.; Curtin, N. J.; Helleday, T., Specific killing of BRCA2-deficient tumours with inhibitors of poly(ADP-ribose) polymerase. 2005, Nature.
Farmer, H.; McCabe, N.; Lord, C. J.; Tutt, A. N.; Johnson, D. A.; Richardson, T. B.; Santarosa, M.; Dillon, K. J.; Hickson, I.; Knights, C.; Martin, N. M.; Jackson, S. P.; Smith, G. C.; Ashworth, A., Targeting the DNA repair defect in BRCA mutant cells as a therapeutic strategy. 2005, Nature.
Pujade-Lauraine, E.; Ledermann, J. A.; Selle, F.; Gebski, V.; Penson, R. T.; Oza, A. M.; Korach, J.; Huzarski, T.; Poveda, A.; Pignata, S.; Friedlander, M.; Colombo, N.; Harter, P.; Fujiwara, K.; Ray-Coquard, I.; Banerjee, S.; Liu, J.; Lowe, E. S.; Bloomfield, R.; Pautier, P., Olaparib tablets as maintenance therapy in patients with platinum-sensitive, relapsed ovarian cancer and a BRCA1/2 mutation (SOLO2/ENGOT-Ov21): a double-blind, randomised, placebo-controlled, phase 3 trial. 2017, The Lancet. Oncology.
Konstantinopoulos, P. A.; Ceccaldi, R.; Shapiro, G. I.; D'Andrea, A. D., Homologous Recombination Deficiency: Exploiting the Fundamental Vulnerability of Ovarian Cancer. 2015, Cancer discovery.
Cambronne, X. A.; Kraus, W. L., Location, Location, Location: Compartmentalization of NAD(+) Synthesis and Functions in Mammalian Cells. 2020, Trends in biochemical sciences.
Lau, C.; Niere, M.; Ziegler, M., The NMN/NaMN adenylyltransferase (NMNAT) protein family. 2009, Frontiers in bioscience.
Gibson, B. A.; Kraus, W. L., New insights into the molecular and cellular functions of poly(ADP-ribose) and PARPs. 2012, Nature reviews. Molecular cell biology.
Gupte, R.; Liu, Z.; Kraus, W. L., PARPs and ADP-ribosylation: recent advances linking molecular functions to biological outcomes. 2017, Genes & development.
Maya-Mendoza, A. et al. High speed of fork progression induces DNA replication stress and genomic instability. Nature 559, 279-284 (2018).
Hanzlikova, H. et al. The Importance of Poly(ADP-Ribose) Polymerase as a Sensor of Unligated Okazaki Fragments during DNA Replication. Molecular Cell 71, 319-331.e3 (2018).
Patel, P. S., Algouneh, A. & Hakem, R. Exploiting synthetic lethality to target BRCA1/2-deficient tumors: where we stand. Oncogene 2021 40:17 40, 3001-3014 (2021).
Gogola, E., Rottenberg, S. & Jonkers, J. Resistance to PARP Inhibitors: Lessons from Preclinical Models of BRCA-Associated Cancer. https://doi.org/10.1146/annurev-cancerbio-030617-050232 3, 235-254 (2019).
Christ, L. et al. (2017) ‘Cellular functions and molecular mechanisms of the ESCRT membrane-scission machinery’, Trends in biochemical sciences. Elsevier, 42(1), pp. 42-56.
Yazinski, S. A. et al. (2017) ‘ATR inhibition disrupts rewired homologous recombination and fork protection pathways in PARP inhibitor-resistant BRCA-deficient cancer cells’, Genes & development. Cold Spring Harbor Lab.
Kusumanchi, P., Zhang, Y., Jani, M. et al. Nicotinamide mononucleotide adenylyltransferase2 overexpression enhances colorectal cancer cell-kill by Tiazofurin. Cancer Gene Ther 20, 403-412 (2013).
Frey, N., Tortola, L., Egli, D. et al. Loss of Rnf31 and Vps4b sensitizes pancreatic cancer to T cell-mediated killing. Nat Commun 13, 1804 (2022).
Szymańska E, Nowak P, Kolmus K, Cybulska M, Goryca K, Derezińska-Wołek E, Szumera-Ciećkiewicz A, Brewińska-Olchowik M, Grochowska A, Piwocka K, Prochorec-Sobieszek M, Mikula M, Miączyńska M. Synthetic lethality between VPS4A and VPS4B triggers an inflammatory response in colorectal cancer. EMBO Mol Med. 2020 Feb 7;12(2):e10812.

What is claimed is:

1. A method of treating HRD cancer in a subject in need thereof, comprising administering to the subject a therapeutically effective amount of a VPS4B inhibitor.

2. The method according to claim 1, wherein the cancer is BRCA-associated cancer

3. The method according to claim 1, wherein the cancer is chemo-resistance BRCA-associated cancer.

4. The method according to claim 1, wherein the cancer is PARPi resistant BRCA-associated cancer or cisplatin resistant BRCA-associated cancer, including those with somatic reversion of the BRCA mutation and HR restoration.

5. The method according to claim 1, wherein said VPS4B inhibitor is a small organic molecule, a polypeptide, an aptamer, an oligonucleotide or an antibody.

6. The method according to claim 1, wherein said VPS4B inhibitor is an antisense oligonucleotide, a siRNA, a shRNA, a DNA aptamer or a RNA aptamer.

7. The method according to claim 1 in combination with a PARP inhibitor.

8. The method according to claim 7, wherein the PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

9. The method according to claim 1 in combination with cisplatin or a Polθ inhibitor such as novobiocin.

10. The method according to claim 1 in combination with a compound selected from the group consisting of PARP inhibitor (PARPi), NMNAT1 inhibitor, SIRT6 inhibitor and/or Polθ inhibitors.

11. A method of treating HRD cancer in a subject in need thereof, comprising administering to the subject a pharmaceutical composition comprising a VPS4B inhibitor and a pharmaceutical acceptable carrier.

12. The method according to claim 11, wherein the cancer is BRCA-associated cancer.

13. The method according to claim 11, wherein the cancer is chemo-resistance BRCA-associated cancer.

14. (canceled)

15. The method according to claim 11, wherein the cancer is PARPi resistant BRCA-associated cancer or cisplatin resistant BRCA-associated cancer, including those with somatic reversion of the BRCA mutation and HR restoration.

16. The method according to claim 11, wherein said VPS4B inhibitor is a small organic molecule, a polypeptide, an aptamer, an oligonucleotide or an antibody.

17. The method according to claim 11, wherein said VPS4B inhibitor is an antisense oligonucleotide, a siRNA, a shRNA, a DNA aptamer or a RNA aptamer.

18. The method according to claim 11, wherein said pharmaceutical composition is administered in combination with a PARP inhibitor.

19. The method according to claim 17, wherein the PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

20. The method according to claim 11, wherein said pharmaceutical composition is administered in combination with cisplatin or a Polθ inhibitor such as novobiocin.

21. The method according to claim 11, wherein said pharmaceutical composition is administered in combination with a compound selected from the group consisting of PARP inhibitor (PARPi), NMNAT1 inhibitor, SIRT6 inhibitor and/or Polθ inhibitors.
